(19) United States
(12) Patent Application Publication
(10) Pub. No.: US 2026/0090747 A1
(43) Pub. Date: Apr. 2, 2026

(54) ADJUSTING GAZE TARGETS

(71) Applicant: NeuraLight Ltd., Tel Aviv (IL)

(72) Inventors: Rotem Zvi BAR-OR, Boulder, CO (US); John Michael ROZMUS, Manor, TX (US); Vladimir ANISIMOV, Uster (CH)

(21) Appl. No.: 18/899,840

(22) Filed: Sep. 27, 2024

(51) Int. Cl. A61B 5/16; A61B 3/00; A61B 3/14; G06T 7/00; G06T 7/50; G06T 7/70; G06T 15/20; G06T 17/00

(52) U.S. Cl. CPC A61B 5/163; A61B 3/0091; A61B 3/14; G06T 7/0012; G06T 7/50; G06T 7/70; G06T 15/20; G06T 17/00; G06T 2207/30041; G06T 2207/30201; G06T 2210/41

(57) ABSTRACT

Disclosed herein are system, method, and computer program product embodiments, and/or combinations and sub-combinations thereof, for adjusting a content of a visual stimulus. In some embodiments, an orientation or a location of a face of a user relative to a display is determined. The content of the visual stimulus is then adjusted to compensate for a misalignment between the orientation or the location of the face of the user and the display. The adjusting comprises maintaining an attribute of the visual stimulus as perceived along a line of sight of the user.

### FIELD

The present disclosure is generally directed to adjusting content of a visual stimulus. In particular, the present disclosure relates to adjusting the content of the visual stimulus based on the orientation or the location of a face of a user relative to a display.

### BACKGROUND

Progression of neurological disorders may be determined using minute eye movements. Typically, these eye movements are measured in well-controlled lab settings (e.g., no movements, controlled ambient light, or other such parameters) using dedicated devices (e.g., infrared eye trackers, pupilometers, or other such devices). However, the dedicated devices are challenging to set up, cost prohibitive, or may involve a significant amount of time and effort to create or maintain the controlled lab setup. Such challenges may discourage the continuous monitoring of the progression of neurological disorders. Continuous monitoring may help in early detection, treating, and caring for individuals that suffer from neurological disorders or mental health conditions.

### SUMMARY

Provided herein are system, apparatus, article of manufacture, method and/or computer program product embodiments, and/or combinations and sub-combinations thereof, for efficiently adjusting the content of a visual stimulus. An example embodiment determines an orientation or a location of a face of a user relative to a display and adjusts content of a visual stimulus to compensate for a misalignment between the orientation or the location of the face of the user and the display. The adjusting comprises maintaining an attribute of the visual stimulus as perceived along a line of sight of the user.

Further features of the present disclosure, as well as the structure and operation of various embodiments, are described in detail below with reference to the accompanying drawings. It is noted that the present disclosure is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF DRAWINGS

The accompanying drawings, which are incorporated herein and form part of the specification, illustrate the present disclosure and, together with the description, further serve to explain the principles of the present disclosure and to enable a person skilled in the relevant art(s) to make and use embodiments described herein.

FIG. 1 is a block diagram of a system for adjusting a content of a visual stimulus, according to some embodiments.

FIG. 2A is a schematic that illustrates a gaze response stimulus on a display area, according to some embodiments.

FIG. 2B is a schematic that illustrates a gaze response stimulus on a display area, according to some embodiments.

FIG. 3 is a schematic that shows a Cartesian coordinate system with respect to a display, according to some embodiments.

FIG. 4A is a schematic that illustrates a head pose measurement, according to some embodiments.

FIG. 4B is a schematic that illustrates a pitch angle for the head pose measurement, according to some embodiments.

FIG. 4C is a schematic that illustrates a roll angle for the head pose measurement, according to some embodiments.

FIG. 4D is a schematic that illustrates a yaw angle for the head pose measurement, according to some embodiments.

FIG. 5 is a schematic that illustrates a tilt of a display with respect to a line of sight of a user, according to some embodiments.

FIG. 6A is a schematic that illustrates a vertical displacement of a gaze target, according to some embodiments.

FIG. 6B is a schematic that illustrates a horizontal displacement of a gaze target, according to some embodiments.

FIG. 6C is a schematic that illustrates an adjusted gaze target, according to some embodiments.

FIG. 6D is a schematic that illustrates an adjusted gaze target, according to some embodiments.

FIG. 7 is an example method for determining an oculometric parameter of an eye of the user, according to some embodiments.

FIG. 8 is an example method for adjusting a content of a visual stimulus, according to some embodiments.

FIG. 9 shows a computer system for implementing various embodiments of this disclosure.

FIG. 10 is a schematic that illustrates geometrical parameters for calculating a depth or a range of a face of the user within a field of view of an image sensor, according to some embodiments.

The features of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. Additionally, generally, the left-most digit(s) of a reference number identifies the drawings in which the reference number first appears. Unless otherwise indicated, the drawings provided throughout the disclosure should not be interpreted as to-scale drawings.

### DETAILED DESCRIPTION

Aspects of the present disclosure relate to a system for adjustment of a content of a visual stimulus shown to a user on a display. In particular, the present disclosure relates to adjusting the content of the visual stimulus based on an orientation or a location of a face of the user with respect to the display.

This specification discloses one or more embodiments that incorporate the features of the present disclosure. The disclosed embodiment(s) are provided as examples. The scope of the present disclosure is not limited to the disclosed embodiment(s). Claimed features are defined by the claims appended hereto.

The embodiment(s) described, and references in the specification to “one embodiment,” “an embodiment,” “an example embodiment,” etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

Spatially relative terms, such as “beneath,” “below,” “lower,” “above,” “on,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The term “about,” “approximately,” or the like may be used herein to indicate a value of a quantity that may vary or be found to be within a range of values, based on a particular technology. Based on the particular technology, the terms may indicate a value of a given quantity that is within, for example, 1-20% of the value (e.g., ±1%, ±5%±10%, ±15%, or ±20% of the value).

Embodiments of the disclosure may be implemented in hardware, firmware, software, or any combination thereof. Embodiments of the disclosure may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, and/or instructions may be described herein as performing certain actions. However, it should be appreciated that such descriptions are merely for convenience and that such actions in fact result from computing devices, processors, controllers, or other devices executing the firmware, software, routines, instructions, etc. In the context of computer storage media, the term “non-transitory” may be used herein to describe all forms of computer readable media, with the sole exception being a transitory, propagating signal.

As noted in the Background section above, various medical conditions including neurological disorders may be determined using minute eye movements. Eye movements may include the process of measuring either a point of gaze (where the user is looking) or an angle of a line of sight of an eye relative to a head of the user.

Typically, these eye movements are measured in well-controlled lab settings (e.g., no movements, controlled ambient light, or other such parameters) using dedicated devices (e.g., infrared eye trackers, pupilometers, or other such devices). However, setting up and maintaining such a controlled test environment may be extremely costly, and may require a significant amount of time and effort. Furthermore, because there exist only a limited number of such well-controlled lab settings, it may be difficult to schedule appointments and/or travel thereto.

To make the benefits of the detection and treatment of neurological conditions and other mental health conditions, it would be desirable to make eye movement measurements available at low cost by, for example, using the ubiquitous cameras included in smartphones, tablets, laptop computers, desktop computers and the like, to observe the behavior of eyes in response to a visual stimulus. Using the eye movement measurements, digital markers indicative of a neurological condition or a mental health condition may be determined.

In some aspects, an eye gaze direction versus time for directed tasks (e.g., saccades, smooth pursuit, long fixation) may be measured. As discussed above, eye gaze is typically measured in a controlled setting where the face of the user is mechanically aligned to the display. Separate horizontal and vertical measurements are typically accomplished by a subject directly facing the center of the display with no tilt of the head or display-neither up/down nor left/right. A chin and forehead rest may be used to securely align a subject's face with the display showing the sequence of gaze targets. The mechanical alignment may be an inconvenience for the user.

As discussed above, monitoring the progress of neurological conditions at home provides several advantages. However, conventional equipment used to diagnostic, classify, and monitor neurological conditions in a laboratory setup are expensive and inconvenient to replicate in a home setup. Consumer electronic devices (e.g., a laptop, a smartphone, a personal computer) may comprise a camera and a display and are accessible for individuals. However, due to the lack of conventional mechanical alignments, the face of the user may not be aligned with the display and/or camera. Thus, oculometric parameters determined based on images captured by the camera may not be accurate due to the misalignment among the display, the user, and/or the camera. Embodiments described herein correct for the position of the camera relative to the display and for vertical and/or horizontal tilt in a display. In addition, the embodiments described herein correct for roll in a head pose of a subject with high precision, which in turn provides accurate oculometric parameters. In some aspects, the embodiments described herein correct the position of gaze targets in a display while measuring oculometric parameters that are used in identifying neurological conditions or mental health conditions.

Embodiments described herein adjust the content of the display outputting the visual stimulus to dynamically compensate for misalignment between the orientation or location of a face of the user and a display. The content of a display associated with measuring gaze is adjusted so that the amount of movement of a subject's eyes relative to the subject's head is measured accurately, regardless of the orientation or location of the head with respect to the display or to the field of view of an image sensor that is measuring that movement.

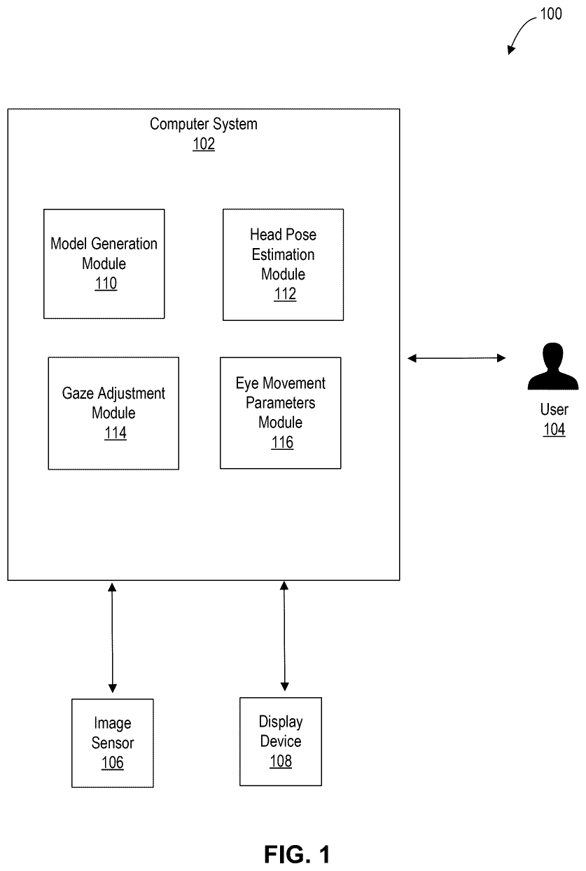

FIG. 1 is a block diagram of a system 100 for adjusting a content of a visual stimulus (e.g., a position of a gaze target on the display), according to some embodiments. System 100 may include a computer system 102, an image sensor 106, and a display device 108.

Display device 108 may be a device that is capable of rendering images generated or acquired by computer system 102 such that a user 104 may visually perceive them. Display device 108 may include a display screen integrated with computer system 102 (e.g., an integrated display of a smartphone, a tablet computer, or a laptop computer) or a monitor separated from but communicatively coupled to computer system 102 (e.g., a monitor connected to a desktop computer via a wired connection) or a projector system (e.g., a projection screen and a projector comprising a light source). Display device 108 may also comprise display panels of a standalone or tethered extended reality headset. In some aspects, display device 108 may display a plurality of frames generated by computer system 102. In some aspects, the plurality of frames may be received by display device 108 via a network.

The network may be a telecommunications network, such as a wired or wireless network. The network can span and represent a variety of networks and network topologies. For example, the network can include wireless communication, wired communication, optical communication, ultrasonic communication, or a combination thereof. For example, satellite communication, cellular communication, Bluetooth, Infrared Data Association standard (IrDA), wireless fidelity (WiFi), and worldwide interoperability for microwave access (WiMAX) are examples of wireless communication that may be included in the network. Cable, Ethernet, digital subscriber line (DSL), fiber optic lines, fiber to the home (FTTH), and plain old telephone service (POTS) are examples of wired communication that may be included in the network. Further, the network can traverse a number of topologies and distances. For example, the network can include a direct connection, personal area network (PAN), local area network (LAN), metropolitan area network (MAN), wide area network (WAN), or a combination thereof.

In some aspects, image sensor 106 may be an optical device that is capable of capturing and storing images and videos. Image sensor 106 may comprise, for example, a digital camera that captures images and videos via an electronic image sensor. Image sensor 106 may be integrated with computer system 102 (e.g., an integrated camera of a smartphone, a tablet computer, or a laptop computer) or part of a device that is separate from but communicatively coupled to computer system 102 (e.g., a USB camera or webcam connected to a desktop computer via a wired connection or the network). In some aspects, image sensor 106 may be integrated with display device 108. In some aspects, image sensor 106 may be coupled to display device 108. In one example, image sensor 106 may be a video camera. In some aspects, image sensor 106 may transmit the captured images or videos via the network.

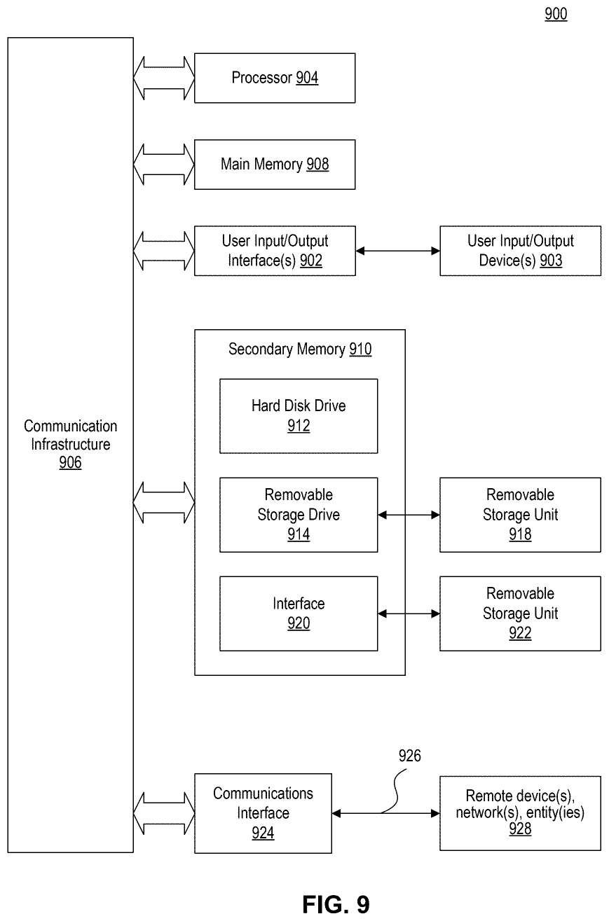

As described previously herein, computer system 102 may be a mobile device, a laptop computer, a desktop computer, a tablet, or other type of electronic device as would be appreciated by a person of ordinary skill in the art. For example, computer system 102 may be a mobile device that comprises image sensor 106 and display device 108. In some aspects, computer system 102 may operate on one or more servers and/or databases. The servers may be a variety of centralized or decentralized computing devices. For example, a server may be grid-computing resources, a virtualized computing resource, peer-to-peer distributed computing devices, or a combination thereof. The servers may be centralized in a single room, distributed across different rooms, distributed across different geographic locations, or embedded within the network. In some embodiments, computer system 102 may be implemented using computer system 900 described with reference to FIG. 9. Computer system 102 may provide a cluster computing platform or a cloud computing platform to perform eye movement measurements based on image frames received or acquired from image sensor 106. Computer system 102 may determine an orientation of a face of user 104 relative to display device 108 based on the image frames. Computer system 102 may adjust a content of a visual stimulus to compensate for a misalignment between the orientation or location of the face of the user and display device 108 before outputting the content to user 104 and before performing the eye movement measurements.

User 104 may be a person interacting with computer system 102. In some embodiments, user 104 may be the person or the subject undergoing oculometric testing or monitoring. The testing may include determining an oculomotor ability of user 104. Oculometric testing may include determining a response of the eye to a particular stimulus (e.g., a visual stimulus). Computer system 102 may determine one or more parameters that are indicative of where the eyes are focused or looking. Computer system 102 may determine different types of parameters such as fixation, saccade, pursuit, or other gaze parameters. In some aspects, fixation is defined as the stability of eye movement while inspecting a specific unmoving area of the stimulus. Saccade is defined as a rapid eye movement between inspection areas of the stimulus. In some aspects, pursuit is defined as a smooth eye movement fixated on a moving area of the stimulus. These are only examples and other types of oculometric tests may be applied to user 104. These eye movement parameters may be used in generating various digital markers (e.g., digital biomarkers).

In some aspects, user 104 may be a person interacting with a virtual reality system (e.g., playing a virtual reality game) where a reaction of the user to a stimulus is determined and used to control one or more parameters of the virtual reality system (e.g., one or more movements in the virtual reality game).

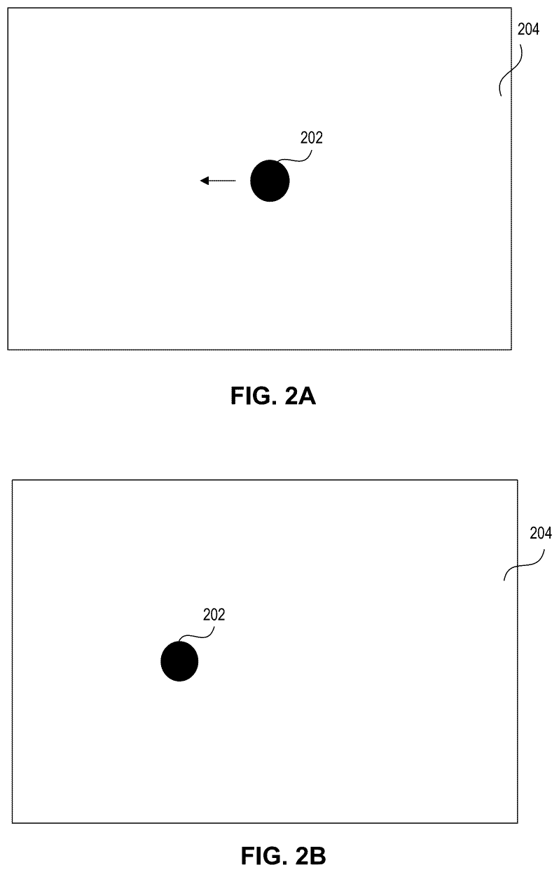

As described previously herein, computer system 102 may be used to determine an oculomotor ability of user 104. Computer system 102 may transmit to display device 108 one or more frames corresponding to the visual stimulus. For example, user 104 may be told to gaze directly at a fixation point on the display as the display is instantaneously switched from the first fixation point to the second (referred to as a gaze response stimulus or a saccade test) as described in relation to FIGS. 2A and 2B. Image sensor 106 may capture an image of the face of the user 104 while the plurality of frames are displayed on display device 108. In order to maintain the accuracy of the measurement, the content of the visual stimulus is adjusted based on the orientation or location of the face of the user. The adjusting comprises maintaining an attribute of the visual stimulus (e.g., shape, displacement distance and orientation) as perceived along a line of sight of the user. To determine the orientation or location of the face of the user, image sensor 106 may record a video of user 104. Computer system 102 may acquire the video from image sensor 106. The video may include a plurality of image frames. In some aspects, computer system 102 may determine the orientation or location of the face of the user using one or more image frames from the plurality of image frames.

In some aspects, saccade tests may include measuring a horizontal saccade and a vertical saccade. Horizontal saccade and vertical saccade may be measured separately as they are associated with separate disorders. A principal reason for this is that distinctly different brain areas are involved in horizontal and vertical saccades. As described above, a saccade is a rapid movement of the eye between two fixation points. During a saccade test, a subject is told to gaze directly at the gaze target on the display as the display is instantaneously switched from the first pattern to the second. A full test sequence will typically include multiple similar images in sequence with the gaze target appearing in a different location in each image with all the different locations spanning much a display area of display device 108. FIG. 2A and FIG. 2B illustrate a horizontal saccade test.

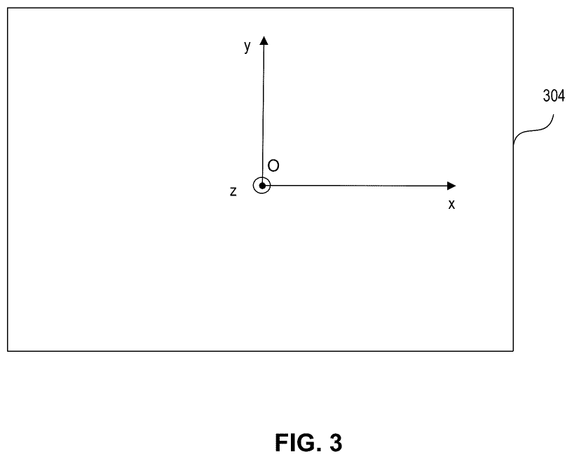

FIG. 2A illustrates a gaze response stimulus displayed on display device 108 at the start of the gaze tracking test such as a saccade test, according to some embodiments. FIG. 2B illustrates the gaze response stimulus displayed on display device 108 at the end of the gaze tracking test, according to some embodiments. A saccade is a rapid movement of the eye between two fixation points. The saccade test measures the ability of a subject to move the eye (or eyes) from one fixation point to another in a single, quick movement. In some aspects, the gaze response test may comprise displaying a first image on display device 108. The first image may include a target 202 (e.g., a dot) on a background 204. The background can be a solid background (a solid uniform color). Target 202 may have a display attribute different from background 204, for example, a different color or intensity. Target 202 may be displayed at a first position. In a second image displayed on display device 108, target 202 can be displayed at a second position as shown in FIG. 2B. Second position may correspond to an horizontal displacement of target 202 in a Cartesian coordinate system shown in FIG. 3.

In a vertical saccade test, target 202 is moved in a vertical direction in a Cartesian coordinate system shown in FIG. 3. In other tests, target 202 may move along a vertical direction and/or a horizontal direction. Target 202 may be moved from left to right, from right to left, from up to down, from down to up, or in a diagonal direction. In some aspects, target 202 may move in a circle. Target 202 may move at different speeds in a smooth movement or abruptly. The movement direction, speed, and other attributes of the gaze tracking test may be selected based on the desired oculometric parameters.

FIG. 3 is a schematic that shows a Cartesian coordinate system with respect to a display 304, according to some embodiments. In some aspects, an origin of the Cartesian coordinate system may correspond to a center of display 304. The vertical direction may correspond to the y-axis in the Cartesian coordinate system. That is, the y-axis may be parallel to the vertical edges of the display. The horizontal direction may correspond to the x-axis in the Cartesian coordinate system. That is, the x-axis may be parallel to the horizontal edges of the display. The z-axis is perpendicular to the display. The Cartesian coordinate system may be used to define the location of gaze targets on display device 108. As would be understood by one or ordinary skills in the art, other coordinate systems may be used (e.g., a Cartesian coordinate system having an origin at a corner of display 304).

Referring back to FIG. 1, computer system 102 may comprise a model generation module 110, a head pose estimation module 112, a gaze adjustment module 114, and an eye movement parameters module 116.

In some aspects, model generation module 110 may process the video stream from image sensor 106 and generate a three-dimensional model (3D model) of the face of user 104 located within the field of view of image sensor 106. The 3D model is synchronized to timestamps of the video stream from image sensor 106. Model generation module 110 may generate the three-dimensional model from one or more image frames extracted from the video stream. In some aspects, the 3D model of a face may be obtained from two-dimensional (2D) video images or a single image from image sensor 106 using artificial intelligence techniques including using neural networks. In some aspects, a deep neural network may be trained to learn a face identity model both in shape and appearance and to reconstruct the 3D face as described in FML: Face Model Learning from Videos, Tewari et al, 2019. The deep neural network may be trained using a big dataset of unconstrained images that includes multiple images of each subject (e.g., monocular videos). In some aspects, the 3D model of the face may be generated using a single image as described in 3D Face Reconstruction from a Single Image using a Single Reference Face Shape, Kemelmacher-Schlizerman and Basri, 2011. The 3D model may be generated using the single image and a single reference 3D model of either a different individual or a generic face. The 3D model may be generated based on shading information in the single image.

In some aspects, model generation module 110 may determine a depth or range of the face of user 104 within the field of view of image sensor 106. In some aspects, model generation module 110 may determine the depth using a diameter of the iris in pixels. The observed diameter d of the iris in pixels may be determined based on an image frame of the face of the user that includes an eye region. For example, using computer vision techniques, the location of the eye may be identified in the image frame. Then, computer system 102 may fit a circle to the iris-sclera boundary to measure d.

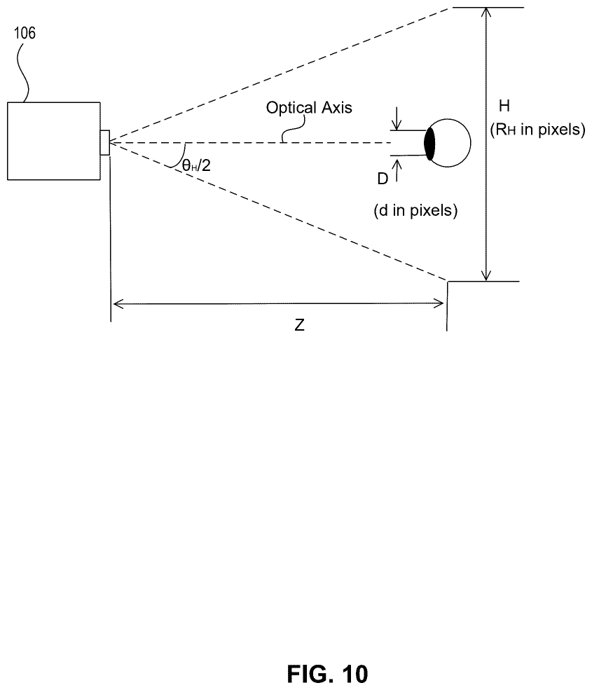

FIG. 10 is a schematic that illustrates geometrical parameters for calculating a depth or a range of a face of the user within a field of view of image sensor 106, according to some embodiments. The physical iris diameter has a small variation in the human population with a size of about 11.9 mm+/−15% (1^{st} and 99^{th} percentile limits). The horizontal resolution in pixels, R_{H}, vertical resolution in pixels, R_{V}, horizontal angular field of view, θ_{H}, and vertical angular field of view, θ_{V}, of image sensor 106 may be known at the outset (e.g., settings) or may be determined (e.g., retrieved). In some aspects, computer system 102 may retrieve a configuration file associated with image sensor 106 to determine R_{H} and θ_{H} of image sensor 106. In some aspects, computer system 102 may send an application programming interface (API) request (e.g., API call) to image sensor 106 to retrieve at least R_{H} and θ_{H} of image sensor 106. A sufficiently accurate estimate of the depth (z dimension) may be obtained using d, θ_{H}, the median physical iris diameter, D=11.9 mm=1.19 cm, and R_{H} of image sensor 106 as follows:

$$z = \frac{\frac{{R}_{H}}{2} \times \frac{D}{d}}{tan \frac{{θ}_{H}}{2}}$$

(as illustrated in FIG. 10.)

In some aspects, when a more accurate estimation of depth or range is desired, a convolutional neural network may be trained on a large dataset of face views to determine the distance between the face of the user and image sensor 106 as described, for example, in High-Accuracy Facial Depth Models derived from 3D Synthetic Data, Khan et al, 2020.

In some aspects, image sensor 106 may be mounted above display device 108. Image sensor 106 may be positioned so that its optical axis passes through the approximate center of a subject's face (that is, in a downward direction). Model generation module 110 may obtain the distance between image sensor 106 and the origin O of display device 108 in FIG. 3, and the angle between the optical axis of image sensor 106 and the z axis in FIG. 3. In some aspects, this distance (the distance between image sensor 106 and the origin O of display device 108) and angle (between the optical axis of image sensor 106 and the z axis) may be retrieved from a specifications file of display device 108. In another aspect, computer system 102 may send an API request (e.g., API call) to obtain this distance and angle. In some aspects, this distance and angle may be input by user 104.

Using the obtained distance and angle, and the 3D face model located in the field of view of image sensor 106 that is synchronized to the timestamps of the video stream from image sensor 106, model generation module 110 may generate from the video stream of image sensor 106 a synthesized video of the face of the user as it would appear to a virtual camera having the same R and θ as image sensor 106 and located at the center of the display at (x,y,z)=(0,0,0) (origin O in FIG. 3) and having an optical axis that coincides with the z axis of FIG. 3. Having a good 3D face model located in time and space means that one can calculate what the face will look like from any angle at any time.

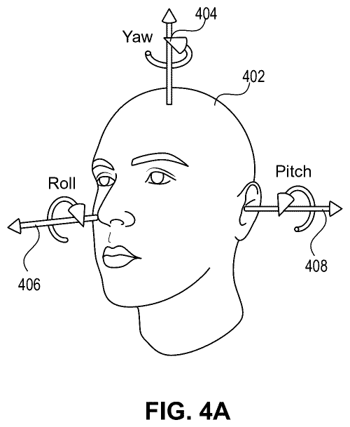

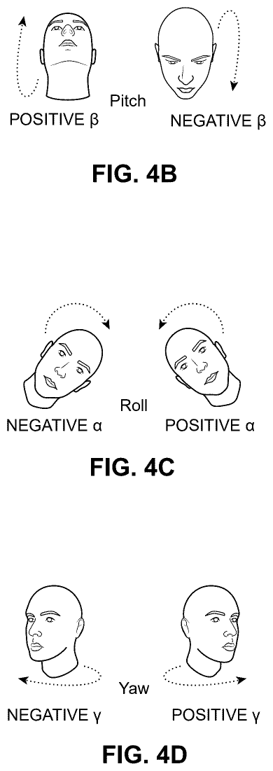

Based on the synthesized video stream, head pose estimation module 112 may determine the orientation or location of the face of the user. In some aspects, the orientation of the face of the user may also be referred to as a head pose. In some aspects, the orientation of the face of the user may be measured using three Euler angles (α,β,γ) as illustrated in FIG. 4A. The Euler angles may be used to describe the orientation of the head with respect to a fixed coordinate system.

FIG. 4A is a schematic that illustrates a head pose measurement for a head 402 of a user, according to some embodiments. In some aspects, a pitch angle is measured from axis 408. A yaw angle is measured from axis 404 and a roll angle is measured from axis 406. In some aspects, axis 408 (pitch), axis 406 (roll), and axis 404 (yaw) are at right angles to one another and intersect at a point in the middle of head 402. A positive pitch angle β and a negative pitch angle β are shown in FIG. 4B. A positive roll angle α and a negative roll angle α are shown in FIG. 4C. A positive yaw angle γ and a negative yaw angle γ are shown in FIG. 4C.

In some aspects, an ideal alignment of face to the display (i.e., to obtain accurate measurements) may have the z-axis on the same line as axis 406 (roll), the y-axis parallel to axis 404 (yaw), and the x-axis parallel to axis 408 (pitch). With this alignment, the head pose as it might be viewed from a virtual camera at the origin of the display at (x,y,z)=(0,0,0) is (α,β,γ)=(0,0,0).

In some aspects, head pose estimation module 112 may determine the head pose using a deep learning neural network as described in Deep Learning for Head Pose Estimation: A Survey, Asperti & Fillipini, 2023. In some aspects, the adjusted video stream obtained from model generation module 110 may be input to the deep learning neural network in order to generate the head pose as it would appear from the origin of the (x,y,z) coordinate system of the display.

In some aspects, the head pose may be at a nominal, neutral position relative to the subject's body. But rotation of display device 108 may occur (e.g., when display device 108 is integrated with a tablet). The rotation of display device 108 may cause the head pose to appear different than (α,β,γ)=(0,0,0) when considered from the perspective of a virtual camera at (x,y,z)=(0,0,0). A rotation of display device 108 through an angle α is equivalent to a roll of the head having angle α when considered from the perspective of a virtual camera at (x,y,z)=(0,0,0) as illustrated in FIG. 4C.

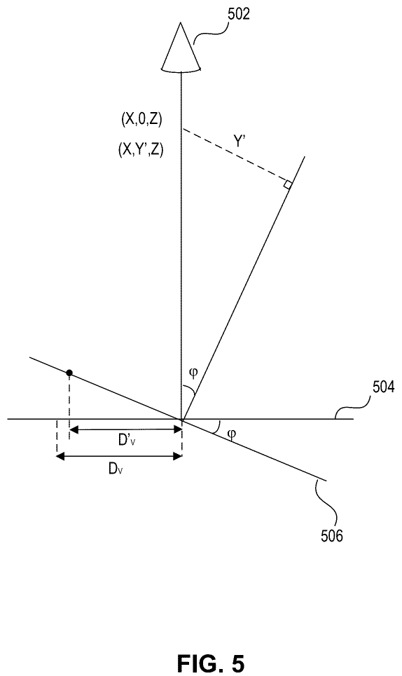

Based on the location of the face in the (x,y,z) coordinate system of the display (FIG. 3), gaze adjustment module 114 may adjust the content of the visual stimulus for a tilt of display device 108. FIG. 5 illustrates an example of tilt in the vertical direction (around the x axis). Consider a face located with the point centered between the two eyes at (x, 0, z). A virtual camera at (x,y,z)=(0,0,0) with optical axis coinciding with the z axis will perceive a vertical tilt having angle φ as a translation of the face location in the y direction having magnitude y′:

$$φ = {sin}^{- 1} \frac{{y}^{′}}{z} \cdot {y}^{′}$$

is a linear physical distance in the plane having depth coordinate z. But with respect to the virtual camera, the translation in number of pixels is yₚ.

$${y}^{′} = {y}_{p} \times \frac{V}{{R}_{V}} ,$$

where R_{V} is the vertical resolution of image sensor 106 in pixels (as defined above) and V is linear physical distance of the full vertical field of view at depth coordinate z. V may be determined using

$$V = 2 \times tan ( \frac{{θ}_{V}}{2} ) \times z ,$$

where θ_{V} is the vertical angular field of view (as defined above).

In some aspects, gaze adjustment module 114 may adjust contents of a visual stimulus for measuring horizontal and vertical saccades. Gaze adjustment module 114 may also adjust the contents based on a tilt of the display. For example, if φ does not equal zero because of a vertical tilt of the display, the size of a displacement D_{V} will appear to be D_{V}′=D_{V} cos(φ) as illustrated in FIG. 5. In order to maintain the displacement (an attribute of the visual stimulus) as perceived along the line of sight of the user, the displacement is modified by 1/cos(φ) when output to the display having position 506. The displacement as perceived by the user is Dv/cos(φ)×cos(φ)=Dv. Thus, the accuracy of the perceived displacement is preserved even when the display is tilted vertically with respect to the user.

Using an analogous calculation, a tilt of display device 108 in the horizontal direction (around the y axis) having angle σ, may also be corrected. A tilt in any direction may be viewed as a linear superposition of horizontal and vertical tilts. Furthermore, a vertical displacement of a subject's face in the field of view of the virtual camera is equivalent to a vertical tilt. And, a horizontal displacement of a subject's face in the field of view of the virtual camera is equivalent to a horizontal tilt.

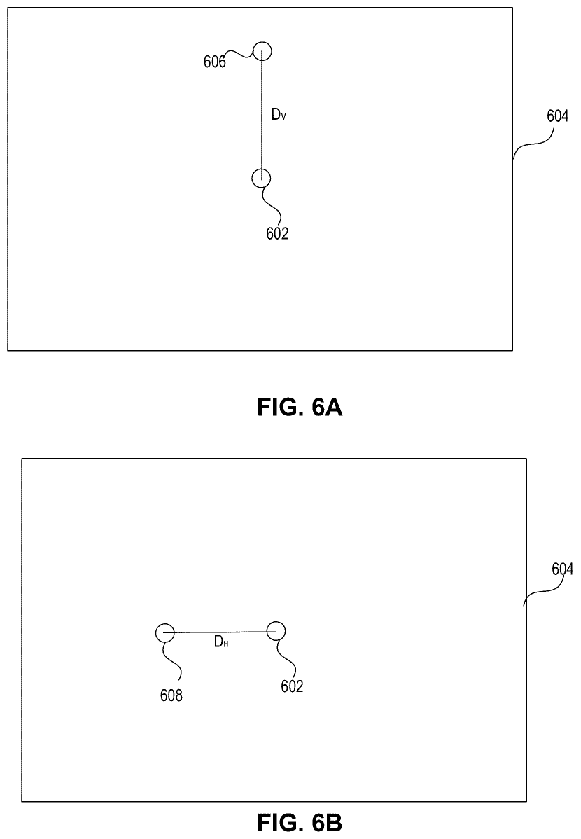

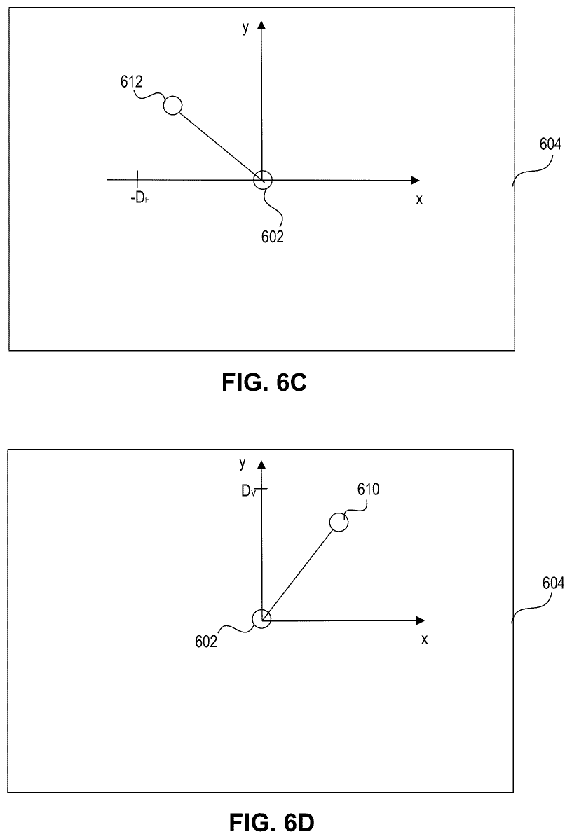

FIG. 6A shows a gaze target for vertical saccade measurements, according to some embodiments. Dᵥ is a vertical displacement to the top to measure vertical saccade when (α,β,γ)=(0,0,0). During vertical saccade measurement, a first image frame may show a target at a first position 602. A second image frame may show the target at a second position 606 on display 604. The vertical displacement between first position 602 and second position 606 may be equal to Dv.

FIG. 6B shows a gaze target for horizontal saccade measurements, according to some embodiments. D_{H} is a horizontal displacement to the left to measure horizontal saccade when (α,β,γ)=(0,0,0). During horizontal saccade measurement, a first image frame may show a target at a first position 602. A second image frame may show the target at a second position 608 on display 604. The horizontal displacement between first position 602 and second position 606 may be equal to D_{H}.

In some aspects, when a does not equal zero (shown in FIG. 4C)—a roll of the head or an equivalent rotation of the display—both horizontal and vertical displacement vectors may be rotated by a degrees in the display in order to maintain direction of displacement relative to the head.

In addition, to maintain perceived size of displacement, the length of the vertical component of a displacement in the display may be corrected by a factor of 1/cos(φ) as discussed above. Similarly, if σ does not equal zero because of a horizontal tilt of the display, the length of the horizontal component of a displacement in the display may be corrected by a factor of 1/cos(σ) to maintain perceived size of displacement.

Accounting for α, φ, and σ, the (x,y,z) coordinates for a corrected position of a target in the display (e.g., the z=0 plane) for measuring a horizontal saccade to the left (negative x coordinate having magnitude D_{H}) is:

$$( - {D}_{H} \frac{cos α}{cos σ} , {D}_{H} \frac{sin α}{cos φ} , 0 ) \qquad ( 1 )$$

FIG. 6C shows the adjusted gaze position for horizontal saccade measurements, according to some embodiments. A gaze target is shown at position 612 on display 604 during horizontal saccade measurements. By outputting the gaze target at position 612 having coordinates determined using equation (1), the misalignment between the orientation or location of the face of the user and the display is compensated for. Thus, an attribute of the visual stimulus (e.g., the horizontal displacement (length and direction)) as perceived along a line of sight of the user is maintained. Thus, the saccade measurement is accurate even without a mechanical alignment of the subject's face to the display.

Similarly, accounting for α, φ, and σ, the (x,y,z) coordinates for a corrected position in the display (the z=0 plane) of a displaced target for measuring a vertical saccade upward (positive y coordinate having magnitude D_{V}) is:

$$( {D}_{V} \frac{sin α}{cos σ} , {D}_{V} \frac{cos α}{cos φ} , 0 ) \qquad ( 2 )$$

FIG. 6D shows the adjusted gaze position for vertical saccade measurements, according to some embodiments. A gaze target is shown at position 610 on display 604 during vertical saccade measurements. By outputting the gaze target at position 612 having coordinates determined using equation (2), the misalignment between the orientation or location of the face of the user and the display is compensated for. Thus, an attribute of the visual stimulus (e.g., the vertical displacement) as perceived along a line of sight of the user is maintained.

The computation of the corrections described above are done with a speed that may compensate for movements of a typical user device (e.g., a smartphone) in real time. In some aspects, built-in sensors for measuring linear acceleration and angular velocity of the user device may be used to ensure that the user device is not moving at a rate that exceeds the speed of the correction computations. In some aspects, computer system 102 may monitor the linear acceleration and angular velocity of display device 108 and image sensor 106. Computer system 102 may abort the saccade measurements when the movement exceeds the speed of the computation of the corrections because the position of the gaze target is not adjusted at the desired rate and the measurements may be inaccurate.

Adjusting the content of the visual stimulus for rotation of the head (or display) or for tilting of the display (or an equivalent displacement of a subject's face) may be generalized to correct every pixel in the display by the use of affine transformations. Vector

$$a = [ \begin{matrix}{X}_{0} \\ {Y}_{0}\end{matrix} ] ,$$

representing the position of any pixel of a content of display device 108 may be adjusted to a new vector location

$$b = [ \begin{matrix}{X}_{1} \\ {Y}_{1}\end{matrix} ]$$

in an adjusted display of the content of the visual stimulus by matrix multiplication with a rotation affine matrix O for rotation and/or a scaling affine matrix S for tilting: b=SOa, where:

$$O = [ \begin{matrix}cos α & sin α \\ - sin α & cos α\end{matrix} ] and S = [ \begin{matrix}1 / cos σ & 0 \\ 0 & 1 / cos α\end{matrix} ] . \\ Thus b = [ \begin{matrix}{X}_{1} \\ {Y}_{1}\end{matrix} ] = [ \begin{matrix}\frac{cos α}{cos σ} & \frac{sin α}{cos σ} \\ \frac{- sin α}{cos φ} & \frac{cos α}{cos φ}\end{matrix} ] [ \begin{matrix}{X}_{0} \\ {Y}_{0}\end{matrix} ] .$$

The x and y coordinates of Equations (1) and (2) are the results of this affine transformation for the cases shown in FIG. 6A, FIG. 6B, FIG. 6C, and FIG. 6D. Display device 108 may have pixels located in a fixed rectangular grid, typically with equal spacing in both x and y directions. The values of these pixels in an adjusted display may be calculated by interpolation, for example, bicubic interpolation, from the full set of b vectors generated from all pixels in the unadjusted display. For example, computer system 102 may determine the values of the pixels in the adjusted display as described above.

In the aspects described above, an initial gaze target is located at the center of the display, which is defined to be an origin with (x,y,z)=(0,0,0). A virtual camera is positioned at this origin. An initial gaze target may be chosen to be at some other location that is not the center of the display. The origin and virtual camera location would then be defined to be at this other location that does not correspond to the center of the display.

In some aspects, content to be presented to the user may be modified based on the orientation or location of the face of the user. The content may be considered as a series or a plurality of images to be output to display device 108. Affine transformations may be applied to the one or more images to spatially transform the one or more images based on the orientation or the location of the face of the user.

In some aspects, a shape corresponding to the gaze target that is output on the display may be modified to maintain an attribute of the gaze target as perceived along a line of sight of the user (e.g., to maintain a square or a circular appearance of the gaze target along the line of sight of the user). In some aspects, data corresponding to the image of the gaze target are transformed using the above equations. For example, the coordinates of the four vertexes of a square are modified using the above equations such that the shape attribute (e.g., square) is maintained as perceived along the line of sight of user 104. The position of each vertex is corrected using equation (1) and equation (2) or all pixels by the corresponding affine transformations. The shape of the gaze target may appear not to be square to a bystander having a line of sight perpendicular to display device 108 but square for user 104.

In some aspects, a gaze target may be a moving target (e.g., moving along a horizontal line from the left side to the right side of display device 108). The position of the gaze target in each frame may be adjusted as described above. In some aspects, the orientation or location of the face of the user may be determined and used to adjust all the positions of the gaze target in display device (e.g., the orientation or location is not updated). In some aspects, the orientation or location of the face of the user may be determined based on each image frame acquired from image sensor 106 or based on a preset frequency (e.g., each predetermined number of acquired image frames of the face of the user). For example, a video stream of the face of user 104 may be captured using image sensor 106. Model generation module 110 may generate the 3D model of the face of the user based on each image frame of the video stream. Based on at least the 3D model, head pose estimation module 112 may determine the orientation or location of the face of the user and the gaze adjustment module 114 may modify the position of the target. In some aspects, the orientation or location of the user may be continuously updated based on acquired image frames and stored in a memory of computer system 102. Gaze adjustment module 114 may retrieve the orientation or location of the face of the user from the memory to determine the position of the gaze target based on the updated orientation or location of the face of the user.

Referring to FIG. 1, eye movement parameters module 116 may analyze the eye region to determine one or more eye movement measurements or other oculometric parameters. Eye movement parameters module 116 may acquire one or more image frames of the face of the user from image sensor 106 while the adjusted content is output on display device 108. Eye movement parameters module 116 may crop from an image frame of the face of the user a region of interest. The region of interest may include the eye of user 104 and an area surrounding the eye. Eye movement parameters module 116 may implement eye segmentation techniques to locate the pupil and/or the iris and determine eye movement measurements. Eye movement parameters module system 102 may determine a saccadic latency based on the eye movement measurements. As discussed above, the saccadic latency is the time from the presentation of the second point of fixation to the start of the saccade. Based on the saccadic latency and/or other oculometric parameters, computer system 102 may determine one or more digital markers that may be indicative of a neurological condition or a mental health condition of user 104 as described in U.S. Pat. No. 12,033,432 entitled “Determining digital markers indicative of a neurological condition” incorporated herein in its entirety.

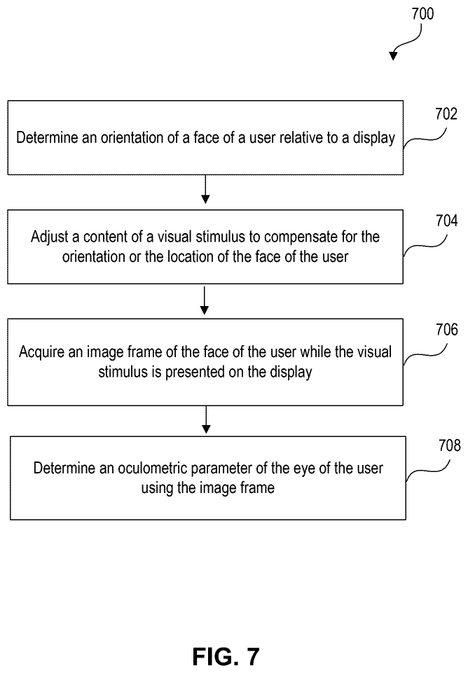

FIG. 7 is an example method for determining an oculometric parameter of an eye of the user, in accordance with an embodiment of the present disclosure. Method 700 may be performed as a series of steps by a computing unit such as a processor. For example, method 700 may be implemented by computer system 102 and/or computer system 900 of FIG. 9. It is to be appreciated that not all steps may be needed to perform the disclosure provided herein. Further, some of the steps may be performed simultaneously, or in a different order than shown in FIG. 7, as will be understood by one of ordinary skill in the art.

Method 700 shall be described with reference to FIG. 1, however, method 700 is not limited to that example embodiment.

At 702, computer system 102 may determine an orientation or a location of a face of a user relative to a display (e.g., display device 108). In some aspects, computer system 102 may determine the orientation and the location of the face of the user relative to the display.

At 704, computer system 102 may adjust a content of a visual stimulus to compensate for the orientation or the location of the face of the user. In some aspects, computer system 102 may adjust the content of the visual stimulus to compensate for a misalignment between the orientation or the location of the face of the user and the display. In some aspects, the adjusting comprises maintaining an attribute of the visual stimulus as perceived along a line of sight of the user.

At 706, computer system 102 may acquire an image frame of the face of the user while the visual stimulus is presented on the display (e.g., display device 108).

At 708, computer system 102 may determine an oculometric parameter of the eye of the user using the image frame. In some aspects, image sensor 106 may capture one or more image frames of the face of the user while the visual stimulus (comprising the adjusted content) is presented on display device 108. In some aspects, the visual stimulus may be a saccade test. The oculometric parameter of the eye of the user using the one or more images frame. In some aspects, computer system 102 may determine one or more digital markers of the user based on the oculometric parameter. The one or more digital markers are indicative of a neurological condition or a mental health condition of the user.

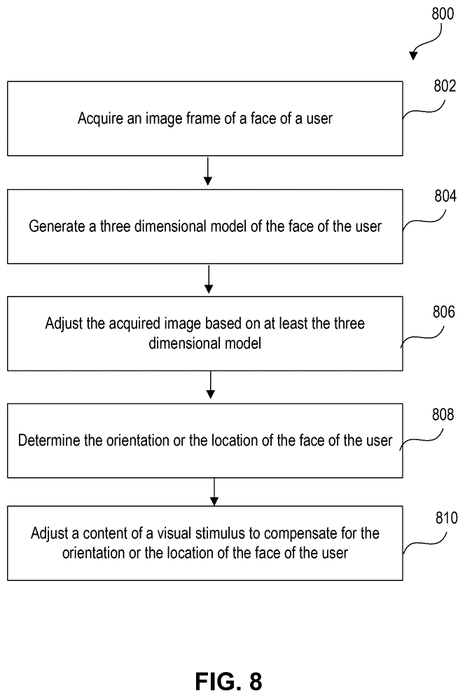

FIG. 8 is an example method for adjusting a content of a visual stimulus, in accordance with an embodiment of the present disclosure. Method 800 may be performed as a series of steps by a computing unit such as a processor. For example, method 800 may be implemented by computer system 102 and/or computer system 900 of FIG. 9. It is to be appreciated that not all steps may be needed to perform the disclosure provided herein. Further, some of the steps may be performed simultaneously, or in a different order than shown in FIG. 8, as will be understood by one of ordinary skill in the art.

At 802, computer system 102 may acquire an image frame of a face of a user. For example, computer system 102 may acquire an image frame from image sensor 106.

At 804, computer system 102 may generate a 3D model of the face of the user using the image frame.

At 806, computer system 102 may adjust the acquired image frame based on at least the 3D model. In some aspects, computer system 102 based on at least the three dimensional model of the face of the user and a position of the image sensor to obtain an adjusted image frame. In some aspects, the position of the image sensor may be determined based on a depth from the image sensor to the face of the user. The computer system 102 may determine the depth based on the diameter of the iris measured in pixels in the image frame as described above. In some aspects, the adjusted image frame comprises the face of the user as visualized by a virtual image sensor positioned at the center of the display.

At 808, computer system 102 may determine the orientation or location of the face of the user using the adjusted image frame. Computer system 102 may determine a roll angle or a tilt angle of the display. The orientation of the face of the user is determined relative to an imaginary line from a center of the display to the center of a head of the user.

At 810, computer system 102 may adjust a content of the visual stimulus to compensate for the orientation or location of the face of the user. In some aspects, the content of the visual stimulus comprises a gaze target. Computer system 102 may adjust the position of the gaze target on the display based on the roll angle or a tilt angle of the display.

FIG. 9 shows a computer system 900, according to some embodiments. Various embodiments and components therein can be implemented, for example, using computer system 900 or any other well-known computer systems. For example, the method steps of FIGS. 7 and 8 may be implemented via computer system 900.

In some aspects, computer system 900 may comprise one or more processors (also called central processing units, or CPUs), such as a processor 904. Processor 904 may be connected to a communication infrastructure or bus 906.

In some aspects, one or more processors 904 may each be a graphics processing unit (GPU). In an embodiment, a GPU is a processor that is a specialized electronic circuit designed to process mathematically intensive applications. The GPU may have a parallel structure that is efficient for parallel processing of large blocks of data, such as mathematically intensive data common to computer graphics applications, images, videos, etc.

In some aspects, computer system 900 may further comprise user input/output device(s) 903, such as monitors, keyboards, pointing devices, etc., that communicate with communication infrastructure 906 through user input/output interface(s) 902. Computer system 900 may further comprise a main or primary memory 908, such as random access memory (RAM). Main memory 908 may comprise one or more levels of cache. Main memory 908 has stored therein control logic (e.g., computer software) and/or data.

In some aspects, computer system 900 may further comprise one or more secondary storage devices or memory 910. Secondary memory 910 may comprise, for example, a hard disk drive 912 and/or a removable storage device or drive 914. Removable storage drive 914 may be a floppy disk drive, a magnetic tape drive, a compact disk drive, an optical storage device, tape backup device, and/or any other storage device/drive. Removable storage drive 914 may interact with a removable storage unit 918. Removable storage unit 918 may comprise a computer usable or readable storage device having stored thereon computer software (control logic) and/or data. Removable storage unit 918 may be a floppy disk, magnetic tape, compact disk, DVD, optical storage disk, and/any other computer data storage device. Removable storage drive 914 reads from and/or writes to removable storage unit 918 in a well-known manner.

In some aspects, secondary memory 910 may comprise other means, instrumentalities or other approaches for allowing computer programs and/or other instructions and/or data to be accessed by computer system 900. Such means, instrumentalities or other approaches may comprise, for example, a removable storage unit 922 and an interface 920. Examples of the removable storage unit 922 and the interface 920 may comprise a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

In some aspects, computer system 900 may further comprise a communication or network interface 924. Communication interface 924 enables computer system 900 to communicate and interact with any combination of remote devices, remote networks, remote entities, etc. (individually and collectively referenced by reference number 928). For example, communication interface 924 may allow computer system 900 to communicate with remote devices 928 over communications path 926, which may be wired and/or wireless, and which may comprise any combination of LANs, WANs, the Internet, etc. Control logic and/or data may be transmitted to and from computer system 900 via communications path 926.

In some aspects, a non-transitory, tangible apparatus or article of manufacture comprising a non-transitory, tangible computer useable or readable medium having control logic (software) stored thereon is also referred to herein as a computer program product or program storage device. This includes, but is not limited to, computer system 900, main memory 908, secondary memory 910, and removable storage units 918 and 922, as well as tangible articles of manufacture embodying any combination of the foregoing. Such control logic, when executed by one or more data processing devices (such as computer system 900), causes such data processing devices to operate as described herein.

Based on the teachings contained in this disclosure, it will be apparent to those skilled in the relevant art(s) how to make and use embodiments of this disclosure using data processing devices, computer systems and/or computer architectures other than that shown in FIG. 9. Embodiments may operate with software, hardware, and/or operating system implementations other than those described herein.

It is to be understood that the phraseology or terminology herein is for the purpose of description and not of limitation, such that the terminology or phraseology of the present disclosure is to be interpreted by those skilled in relevant art(s) in light of the teachings herein.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

While specific embodiments of the disclosure have been described above, it will be appreciated that embodiments of the present disclosure may be practiced otherwise than as described. The descriptions are intended to be illustrative, not limiting. Thus, it will be apparent to one skilled in the art that modifications may be made to the disclosure as described without departing from the scope of the claims set out below.

The foregoing description of the specific embodiments will so fully reveal the general nature of the present disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein.

The breadth and scope of the protected subject matter should not be limited by any of the above-described exemplary embodiments, but should be defined only in accordance with the following claims and their equivalents.

1. A computer-implemented method for adjusting a content of a visual stimulus, comprising:
acquiring one or more image frames of a face of a user;
determining, using one or more processors, an orientation or a location of the face of the user relative to a display based on the one or more image frames of the face of the user; and
adjusting the content of the visual stimulus to compensate for a misalignment between the orientation or the location of the face of the user and the display, wherein the adjusting comprises maintaining an attribute of the visual stimulus as perceived along a line of sight of the user and wherein the attribute of the visual stimulus comprises a length, a direction, or a shape of the visual stimulus.

2. The computer-implemented method of claim 1, wherein the determining the orientation or the location of the face of the user comprises:
generating a three-dimensional model of the face of the user using the one or more image frames of the face of the user, wherein the one or more image frames are acquired via an image sensor;
adjusting the one or more image frames based on at least the three-dimensional model of the face of the user and a position of the image sensor to obtain one or more adjusted image frames; and
determining the orientation or the location of the face of the user using the one or more adjusted image frames.

3. The computer-implemented method of claim 2, further comprising:
determining a depth from the image sensor to the face of the user.

4. The computer-implemented method of claim 2, wherein the one or more adjusted image frames comprises the face of the user as visualized by a virtual image sensor positioned at a center of the display.

5. The computer-implemented method of claim 1, wherein the determining the orientation or the location of the face of the user comprises:
determining a roll angle or a tilt angle of the display.

6. The computer-implemented method of claim 5, wherein the content of the visual stimulus comprises a gaze target, and
wherein the adjusting the content of the visual stimulus comprises:
adjusting a position of the gaze target based on the roll angle or the tilt angle of the display.

7. The computer-implemented method of claim 1, wherein the orientation of the face of the user is determined relative to an imaginary line from a center of the display to the center of a head of the user.

8. The computer-implemented method of claim 1, further comprising:
acquiring another image frame of the face of the user while the visual stimulus is presented on the display;
determining an oculometric parameter of an eye of the user using the another image frame; and
determining one or more digital markers of the user based on the oculometric parameter, wherein the one or more digital markers are indicative of a neurological condition or a mental health condition of the user.

9. The computer-implemented method of claim 1, wherein the visual stimulus comprises a saccade test.

10. A system for adjusting a content of a visual stimulus, comprising:
one or more memories;
at least one processor each coupled to at least one of the memories and configured to perform operations comprising:
acquiring one or more image frames of a face of a user;
determining an orientation or a location of the face of the user relative to a display based on the one or more image frames of the face of the user; and
adjusting the content of the visual stimulus to compensate for a misalignment between the orientation or the location of the face of the user and the display, wherein the adjusting comprises maintaining an attribute of the visual stimulus as perceived along a line of sight of the user and wherein the attribute of the visual stimulus comprises a length, a direction, or a shape of the visual stimulus.

11. The system of claim 10, wherein the determining the orientation or location of the face of the user comprises:
generating a three-dimensional model of the face of the user using the one or more image frames of the face of the user, wherein the one or more image frames is acquired via an image sensor;
adjusting the one or more image frames based on at least the three-dimensional model of the face of the user and a position of the image sensor to obtain one or more adjusted image frames; and
determining the orientation or the location of the face of the user using the one or more adjusted image frames.

12. The system of claim 11, wherein the operations further comprise:
determining a depth from the image sensor to the face of the user.

13. The system of claim 12, wherein the one or more adjusted image frames comprises the face of the user as visualized by a virtual image sensor positioned at a center of the display.

14. The system of claim 10, wherein the determining the orientation or location of the face of the user comprises:
determining a roll angle or a tilt angle of the display.

15. The system of claim 14, wherein the content of the visual stimulus comprises a gaze target, and
wherein the adjusting the content of the visual stimulus comprises:
adjusting a position of the gaze target based on the roll angle or the tilt angle of the display.

16. A non-transitory computer-readable medium having instructions stored thereon that, when executed by at least one computing device, cause the at least one computing device to perform operations comprising:
acquiring one or more image frames of a face of a user;
determining an orientation or a location of the face of the user relative to a display based on the one or more image frames of the face of the user; and
adjusting a content of a visual stimulus to compensate for a misalignment between the orientation or the location of the face of the user and the display, wherein the adjusting comprises maintaining an attribute of the visual stimulus as perceived along a line of sight of the user and wherein the attribute of the visual stimulus comprises a length, a direction, or a shape of the visual stimulus.

17. The non-transitory computer-readable medium of claim 16, wherein the determining the orientation or location of the face of the user comprises:
generating a three-dimensional model of the face of the user using the one or more image frames of the face of the user, wherein the one or more image frames are acquired via an image sensor;
adjusting the one or more image frames based on at least the three-dimensional model of the face of the user and a position of the image sensor to obtain one or more adjusted image frames; and
determining the orientation or the location of the face of the user using the one or more adjusted image frames.

18. The non-transitory computer-readable medium of claim 17, wherein the operations further comprise:
determining a depth from the image sensor to the face of the user.

19. The non-transitory computer-readable medium of claim 17, wherein the one or more adjusted image frames comprises the face of the user as visualized by a virtual image sensor positioned at a center of the display.

20. The non-transitory computer-readable medium of claim 16, wherein the determining the orientation or location of the face of the user comprises:
determining a roll angle or a tilt angle of the display.

21. The computer-implemented method of claim 1, wherein the one or more images are a sequence of images from a video of the user.
